(19) United States
(12) Patent Application Publication
(10) Pub. No.: US 2017/0188825 A1
(43) Pub. Date: Jul. 6, 2017

(54) EXTENDED DEPTH OF FOCUS CONTACT LENS FOR VITREORETINAL SURGERY

(71) Applicant: NOVARTIS AG, Basel (CH)

(72) Inventors: Steven T. Charles, Germantown, TN (US); Ron Smith, Lake Forest, CA (US)

(21) Appl. No.: 14/984,936

(22) Filed: Dec. 30, 2015

(51) Int. Cl. A61B 3/125; A61B 3/13; G02B 27/42; A61F 9/009; G02B 27/00

(52) U.S. Cl. CPC A61B 3/125; A61F 9/009; G02B 27/0075; G02B 27/4216; G02B 27/4211; A61B 3/13

(57) ABSTRACT

A contact lens is usable during ophthalmic surgery, such as vitreoretinal surgery, and includes a diffractive structure that extends depth of focus along an optical axis of the contact lens.

### BACKGROUND

Field of the Disclosure

The present disclosure relates to ophthalmic surgery, and more specifically, to mechanical support of an indirect contact lens by a surgical microscope during vitreoretinal surgery.

Description of the Related Art

In ophthalmology, eye surgery, or ophthalmic surgery, is performed on the eye and accessory visual structures. More specifically, vitreoretinal surgery encompasses various delicate procedures involving internal portions of the eye, such as the vitreous humor and the retina. Different vitreoretinal surgical procedures are used, sometimes with lasers, to improve visual sensory performance in the treatment of many eye diseases, including epimacular membranes, diabetic retinopathy, vitreous hemorrhage, macular hole, detached retina, and complications of cataract surgery, among others.

During vitreoretinal surgery, an ophthalmologist typically uses a surgical microscope to view the fundus through the cornea, while surgical instruments that penetrate the sclera may be introduced to perform any of a variety of different procedures. The surgical microscope provides imaging and optionally illumination of the fundus during vitreoretinal surgery. The patient typically lies supine under the surgical microscope during vitreoretinal surgery and a speculum is used to keep the eye exposed. Depending on a type of optical system used, the ophthalmologist has a given field of view of the fundus, which may vary from a narrow field of view to a wide field of view that can extend to peripheral regions of the fundus.

The optical system to provide the view of the fundus to the surgeon during vitreoretinal surgery may include a special ocular lens, of which various types are typically used, including a direct (plano, flat, or magnifying) contact lens, an indirect non-contact lens, or an indirect contact lens. A contact lens is in physical contact with the cornea and therefore has a concave surface to match the convex surface of the cornea. Typically a small amount of refractive index-matching gel or fluid resides between the cornea and the contact lens to prevent unwanted extraneous interfacial reflections and to protect the cornea from dehydration.

For many types of vitreoretinal surgery using the surgical microscope, the surgeon may desire to have a very wide field of view of the fundus that extends beyond the equator and even out to the ora serrata. For example, plano-concave contact lenses may be used during vitreoretinal surgery to enable visualization of the retina by eliminating the optical effect of corneal curvature. Similarly, a variety of wide angle contact lenses, typically doublets with an air gap, may be used during vitreoretinal surgery to view the peripheral retina and enable viewing of the retina after the vitreous cavity is filled with air instead of fluid in phakic eyes and pseudophakic (having an intraocular lens implanted) eyes.

During vitreoretinal surgery, the patient's head may experience rhythmic up and down movement caused by respiratory motion. In certain operations, such as during macular surgery and inner limiting membrane (ILM) peeling, such motion of the patient's head and eye may make surgery more difficult and less safe, because the image being viewed by the surgeon using a conventional contact lens may periodically vary from being in focus to out of focus as a result of the motion, which is undesirable. Although a smaller aperture may be installed in the light path of the surgical microscope to improve depth of focus, the smaller aperture will also decrease an amount of light entering the eye for viewing during vitreoretinal surgery, which is undesirable for imaging purposes.

### SUMMARY

The disclosed embodiments of the present disclosure provide for illuminating and viewing the interior of the eye during vitreoretinal surgery with extended depth of focus and without relying on a reduced aperture to restrict light levels entering the eye.

In one aspect, a disclosed contact lens is used for performing ophthalmic surgery. The contact lens may include a diffractive structure for extending depth of focus of visible light along an optical axis of the contact lens.

In any of the disclosed embodiments of the contact lens, the contact lens may be selected from a plano-convex lens and a wide angle lens.

In any of the disclosed embodiments of the contact lens, the diffractive structure may be formed on an external surface of the contact lens. In any of the disclosed embodiments, the diffractive structure may be formed on a mating surface of the contact lens that mates with an eye during ophthalmic surgery.

In any of the disclosed embodiments of the contact lens, the contact lens may include a doublet lens, while the diffractive structure may be formed on an interior surface of the doublet lens.

In any of the disclosed embodiments of the contact lens, a focal region of the diffractive structure may correspond to the distance between the contact lens and the retina of an eye when the contact lens is in contact with the eye.

In any of the disclosed embodiments of the contact lens, the contact lens may include optical correction for spherical aberration. In any of the disclosed embodiments of the contact lens, wherein the contact lens may include optical correction for chromatic aberration.

In another aspect, a disclosed method for performing ophthalmic surgery includes positioning a first optical axis of a surgical microscope along a second optical axis of an eye of a patient, and viewing an interior portion of the eye using a contact lens in contact with the eye. In the method, the contact lens may include a diffractive structure for extending depth of focus of visible light along an optical axis of the contact lens.

In any of the disclosed embodiments of the method, the contact lens may be selected from a plano-convex lens and a wide angle lens.

In any of the disclosed embodiments of the method, the diffractive structure may be formed on an external surface of the contact lens.

In any of the disclosed embodiments of the method, the diffractive structure may be formed on a mating surface of the contact lens that mates with an eye during ophthalmic surgery.

In any of the disclosed embodiments of the method, the contact lens may include a doublet lens, and wherein the diffractive structure is formed on an interior surface of the doublet lens.

In any of the disclosed embodiments of the method, a focal region of the diffractive structure may correspond to the distance between the contact lens and the retina of an eye when the contact lens is in contact with the eye.

In any of the disclosed embodiments of the method, the contact lens may include optical correction for spherical aberration. In any of the disclosed embodiments of the method, the contact lens may include optical correction for chromatic aberration.

### BRIEF DESCRIPTION OF THE DRAWINGS

For a more complete understanding of the present invention and its features and advantages, reference is now made to the following description, taken in conjunction with the accompanying drawings, in which:

FIG. 1 is a depiction of an embodiment of a vitreoretinal surgery using a surgical microscope and an extended depth of focus contact lens;

FIG. 2 is a depiction of an embodiment of an extended depth of focus contact lens;

FIG. 3 is a depiction of an embodiment of a diffractive structure used in an extended depth of focus contact lens; and

FIG. 4 is a flow chart of selected elements of a method for performing vitreoretinal surgery using an extended depth of focus contact lens.

### DESCRIPTION OF PARTICULAR EMBODIMENT(S)

In the following description, details are set forth by way of example to facilitate discussion of the disclosed subject matter. It should be apparent to a person of ordinary skill in the field, however, that the disclosed embodiments are exemplary and not exhaustive of all possible embodiments.

As used herein, a hyphenated form of a reference numeral refers to a specific instance of an element and the un-hyphenated form of the reference numeral refers to the collective element. Thus, for example, device ‘12-1’ refers to an instance of a device class, which may be referred to collectively as devices ‘12’ and any one of which may be referred to generically as a device ‘12’.

As noted above, conventional contact lenses used by surgeons during vitreoretinal surgery may result in non-ideal focus due to the combination of having a single focal plane and the unavoidable motion of the patient, and hence the eye of the patient relative to the contact lens. As a result, the image viewed by the surgeon while performing delicate procedures may vary from being in focus to out of focus outside the control of the surgeon or other personnel, which is undesirable. Using a narrowed aperture in the optical system may improve depth of focus, but may also cause an amount of light entering the eye to be reduced, which is undesirable for quality of imaging purposes.

As will be described in further detail, the inventors of the present disclosure have developed an extended depth of focus contact lens for use during vitreoretinal surgery. The extended depth of focus contact lens disclosed herein may be used during vitreoretinal surgery to view interior portions of the eye without having a narrowed aperture that restricts the amount of light entering the eye. The extended depth of focus contact lens disclosed herein may enable sharp viewing of the retina and associated structures during vitreoretinal surgery, without a high degree of sensitivity of the image focus on small movements of the patient.

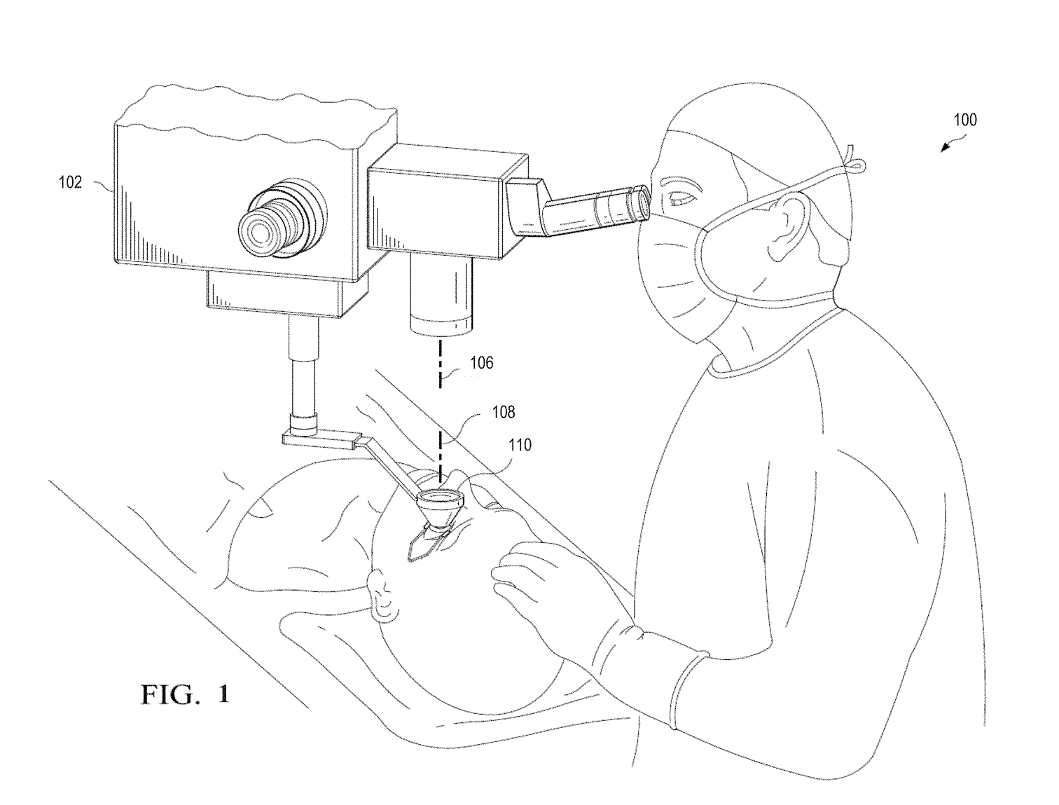

Referring now to the drawings, FIG. 1 illustrates a depiction of an embodiment of a vitreoretinal surgery 100 using a surgical microscope 102 and contact lens 110. Although FIG. 1 is shown with surgical microscope 102 above the patient, it is noted that different orientations of the patient with respect to surgical microscope 102 may be practiced in different embodiments.

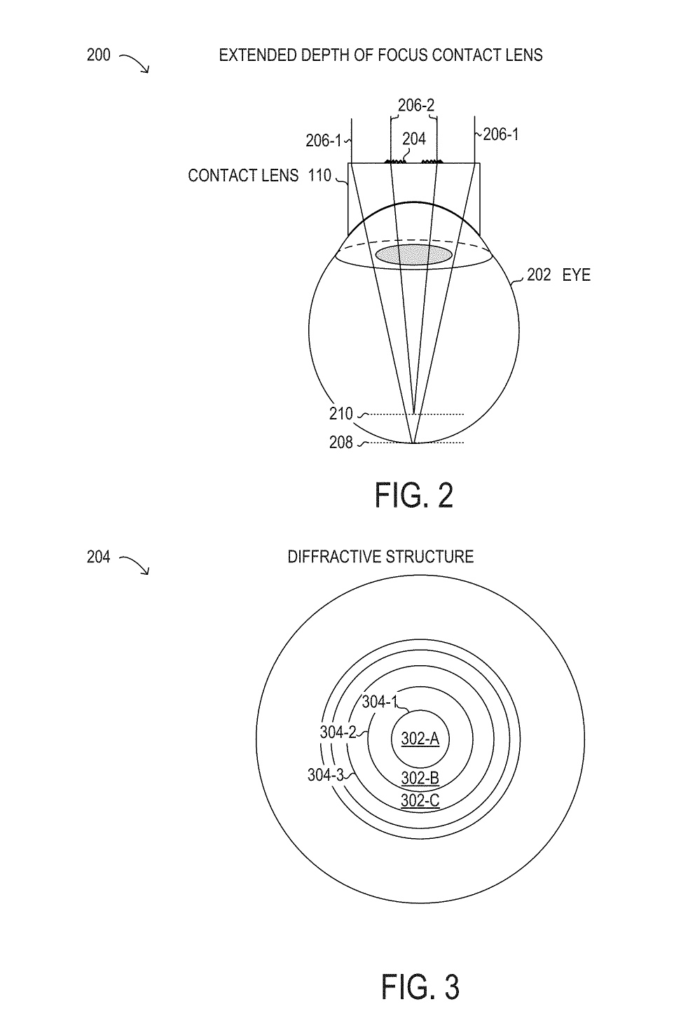

The patient may have an eye exposed using a speculum that enables contact lens 110, to be placed on the eye, typically on the cornea, while the surgeon is viewing the fundus of the patient's eye using surgical microscope 102. Contact lens 110 may be used with an external mechanical support or in a free-standing manner. When contact lens 110 is initially placed on the eye, optical axis 108 of the eye will generally be aligned with optical axis 106 of surgical microscope 102. The objective used with surgical microscope 102 may have a focal length of about 175 mm to 225 mm that focuses on a focal plane of contact lens 110 (see also FIG. 2). It is noted that surgical microscope 102 may provide illumination for the fundus that is projected through contact lens 110.

Contact lens 110 may enable extended depth of focus using a diffractive structure (not visible in FIG. 1, see FIGS. 2 and 3) that is formed on an optical surface of contact lens 110. The optical surface having the diffractive structure formed thereon may be an external surface of contact lens 110, such as a top surface facing surgical microscope 102, as shown, or a mating surface that mates with the eye. The optical surface having the diffractive structure formed thereon may be an internal surface of contact lens 110, for example, when contact lens 110 is a doublet lens with interior optical surfaces.

The diffractive structure may provide a secondary focal plane, in addition to a primary focal plane of contact lens 110 itself without the diffractive structure. The diffractive structure may direct a portion of the incident light to the secondary focal plane as well as to an intermediate region between the primary and secondary focal planes, as described in further detail below. Contact lens 110 may be formed using a suitable optical material, such as glass or quartz, while the diffractive structure may be patterned or machined on the optical surface. A typical diffractive structure may comprise grooves or ridges of a particular height, width, and spacing (i.e., a diffraction grating) to achieve the desired level or degree of extended depth of focus. The diffractive structure may be dimensionally formed for distances typically applicable for ophthalmic surgery, such as vitreoretinal surgery, and for visible wavelengths of light. Specifically, the separation of the focal planes is determined by the spacing of the diffractive elements in the diffractive structure: the larger the spacing, the smaller the distance between the focal planes; the smaller the spacing, the larger the distance between the focal planes. Additionally, a step height for different diffractive zones may be varied to extend the depth of focus of any given focal plane, as described in further detail below.

As a result of the diffractive structure, chromatic dispersion may be observed due to different wavelengths of light being directed away at different directions. Thus, contact lens 110 may be designed as a hybrid lens having both refractive and diffractive optical power, such that the chromatic dispersion due to the diffractive structure is at least partially compensated by the refractive chromatic aberration of contact lens 110. In some embodiments, chromatic dispersion of the diffractive structure may be used as a design variable, in combination with other design factors (such as lens materials, lens thickness, lens radii of curvature, and air thicknesses) to enable contact lens 110 to exhibit lower overall aberrations, extended field angles, and improved depth of focus.

It is noted that in some embodiments, diffractive structure may be an electrically controlled device, such as a liquid crystal switch devices. One example of liquid crystal switch device is an electrically-switchable holographic polymer-dispersed liquid crystal (H-PDLC) grating. The H-PDLC grating may enable an electrically adjustable image plane axial position over a range of N axial positions, where N is the number of gratings in a given grating stack. The grating stack may be situated in an interior portion of contact lens 110, for example when contact lens 110 includes a refractive lens (e.g., a plano-convex lens bonded to N flat gratings bonded to a plano-convex lens to create a convex-convex electrically switchable lens assembly). In other embodiments of the liquid crystal switch device, the diffractive structure may comprise an electrically-controlled liquid crystal phase grating that enables turning the diffractive effect on and off. In this manner, the extended depth of field operation of contact lens 110 may be switched on and off, which may be desirable when the diffractive structure results in decreased resolution.

Referring to FIG. 2, selected elements of an embodiment of an extended depth of focus contact lens system 200 are illustrated. It is noted that FIG. 2 is a schematic diagram and is not drawn to scale. FIG. 2 illustrates the extended depth of focus properties of contact lens 110, as described herein. In FIG. 2, an eye 202 is shown having contact lens 110 placed thereon, such as for performing vitreoretinal surgery as discussed above with respect to FIG. 1. As shown in FIG. 2, contact lens 110 is shown as a plano-convex lens having a diffractive structure 204 formed thereon at a top, external surface that is open to air. It is noted that other types of lenses may be used for contact lens 110, as disclosed herein.

In FIG. 2, light rays 206-1 show how light is directed to a primary focal plane 208 by the optical power of contact lens 110. Light rays 206-2 show how light is directed to a secondary focal plane 210 due to the optical function of diffractive structure 204. Furthermore, diffractive structure 204 may include features (not visible in FIG. 2, see FIG. 3) than enable light to be directed and focused at an intermediate location between secondary focal plane 210 and primary focal plane 208. As a result, system 200 may enable imaging at locations between secondary focal plane 210 and primary focal plane 208 when contact lens 110 is used and may provide improved imaging sensitivity to certain motions of the patient during vitreoretinal surgery that may otherwise cause the image viewed by the surgeon to go out of focus.

Referring to FIG. 3, selected elements of an embodiment of diffractive structures 204 are illustrated. It is noted that FIG. 3 is a schematic diagram and is not drawn to scale. In FIG. 3, diffractive structures 204 are shown as an exemplary embodiment for descriptive purposes to show further details of patterning and forming a surface of contact lens 110 to achieve an extended depth of field. It is noted that in various embodiments, different types of diffractive structures may be used to achieve various kinds of extended depth of field.

In the exemplary embodiment of FIG. 3, diffractive structure 204 may comprise a plurality of concentric, annular diffractive zones 302 separated from one another by a plurality of steps 304. Diffractive zones 302 may serve to generate an image at secondary focal plane 210, as described above with respect to FIG. 2. More particularly, each diffractive zone 302 may be is separated from an adjacent diffractive zone 302 by a step 304, representing a change in a surface level, and material thickness, of contact lens 110. For example, step 304-1 may separate a first diffractive zone 302-A from a second diffractive zone 302-B, while step 304-2 may separate second diffractive zone 302-B from a third diffractive zone 302-C. Other steps and diffractive zones shown in FIG. 3 may be similarly implemented but are not labeled for descriptive clarity.

Due to the change in surface level at step 304, each step 304 may impart a phase delay to incident light passing through at that location, such that the change in thickness of the optical material that contact lens 110 is constructed from (i.e., the depth or height of step 304 in or out of the page of FIG. 3) may be modulated to define the phase delay. As a result of the phase delay of the incident light imparted by steps 304, a portion of the incident light may be directed to an intermediate location between primary focal plane 208 and secondary focal plane 210, resulting in an extended depth of focus that may be observed by a user. Specifically, in the exemplary embodiment shown in FIG. 3, a first phase delay generated by step 304-1, separating first diffractive zone 302-A (the central diffractive zone) from second diffractive zone 302-B, may be different from a second phase delay caused by the step 304-3 and the other nonlabeled steps in FIG. 3, such that a portion of the light incident on the lens is directed to the intermediate location.

In the exemplary embodiment of FIG. 3, diffractive zones 302 comprise a plurality of annular zones whose boundaries are radially located relative to the optical axis 18 in accordance with Equation 1.

rᵢ²=r₀²+2iλf Equation (1)

### In Equation 1:

i denotes a diffraction zone integer (i=0 denotes first diffraction zone 302-A);

λ denotes a design wavelength;

f denotes a focal length corresponding to focal plane 210; and

r₀ denotes a radius of first diffraction zone 302-A.

In some embodiments, the design wavelength λ may be chosen to correspond to green light (˜550 nm) at the center of the visual response. In some particular cases, the radius r₀ may be set to equal to \sqrt{λf}.

As noted, in the exemplary embodiment of FIG. 3, step 304-1 may have a first height, while the remaining steps, including steps 304-2 and 304-3, may all be at a second height that is substantially uniform. For example, the corresponding difference between a first phase delay generated by step 304-1 due to the first height, and a second phase delay generated by each of the other steps due to the second height may be greater than about 1/20 wavelength or (λ/20). In some embodiments, the difference in phase delay is greater than about ¼ wavelength or (λ/4). The wavelength λ may correspond to at least one wavelength in a range of about 400 nm to about 700 nm and may be selected for design purposes.

More generally, the step height H for diffractive structure 204 as shown in FIG. 3 may be given by Equation 2.

$$H = \frac{b λ}{( {n}_{2} - {n}_{1} )} \qquad Equation ( 2 )$$

### In Equation 2:

b denotes the phase height;

λ denotes the design wavelength;

n₂ denotes the refractive index of the lens material; and

n₁ denotes the refractive index of the medium surrounding the lens material.

In the exemplary embodiment of FIG. 3, steps 304 excluding step 304-1 may be substantially uniform and may produce an optical phase delay that results in diffractive structure 204 dividing the incident light approximately equally between focal plane 210 (near focus corresponding to the first order of diffractive structure 204), and focal plane 208 (distance focus corresponding to the zero-th diffraction order. In contrast, step 304-1 separating first diffraction zone 302-A from second diffraction zone 302-B may generate a different phase delay, which causes some of the incident light to be directed to an intermediate location between secondary focal plane 210 and primary focal plane 208. First diffraction zone 302-A may partially contribute to the regular diffractive structure and may accordingly be referred to as a “frustrated diffractive structure” that results in a “frustrated diffraction”. The intermediate location between focal planes 210 and 208 may be referred to as an intermediate focus. In some embodiments, the light convergence at the intermediate focus may result in a duller or less sharp focus than observed at focal planes 210 and 208. In some embodiments, the heights of steps 304 may be apodized, such that the height of steps 304 vary as a function of their radial distance from the optical axis or a center point of diffractive structure 204.

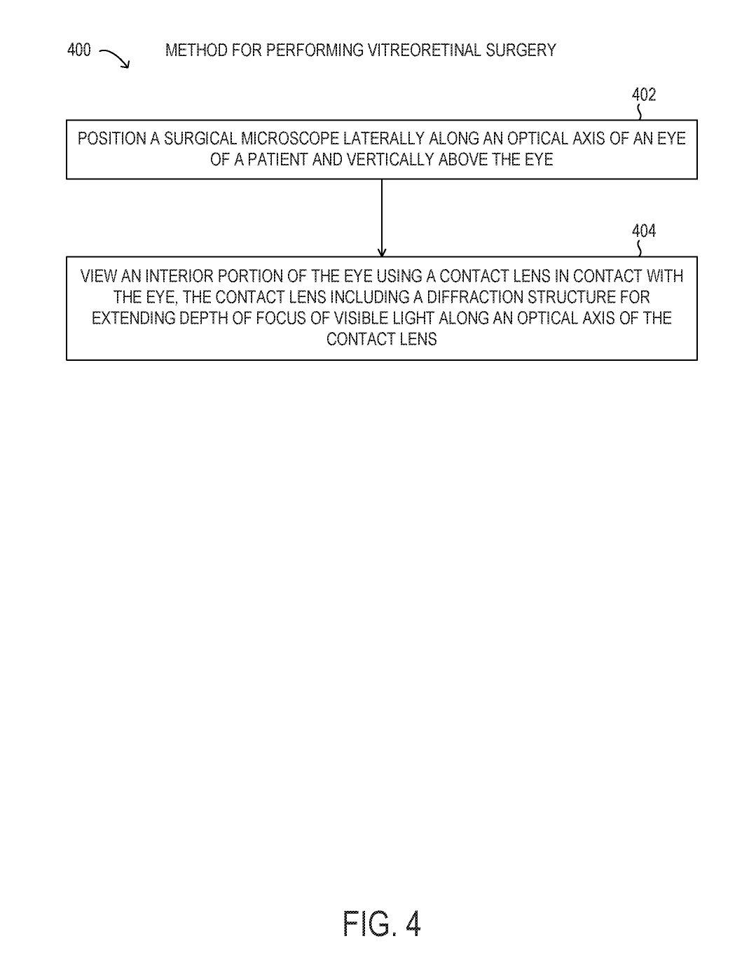

Referring now to FIG. 4, a flow chart of selected elements of an embodiment of a method 300 for performing vitreoretinal surgery, as described herein, is depicted in flowchart form. Method 300 describes steps and procedures for using surgical microscope 100 with contact lens 110 (see FIG. 1) to view the fundus of an eye and to enable further surgical procedures based on the view of the fundus. It is noted that certain operations described in method 400 may be optional or may be rearranged in different embodiments. Method 300 may be performed by a surgeon or by other medical personnel. In some embodiments, at least certain portions of method 400 may be automated, for example using servo-mechanical control associated with certain aspects of the surgical microscope, such as raising or lowering the surgical microscope.

Method 400 may begin, at operation 402, by positioning a surgical microscope laterally along an optical axis of an eye of a patient and vertically above the eye. In certain embodiments of operation 402, the patient is moved relative to the surgical microscope. Then, at operation 404, an interior portion of the eye may be viewed using a contact lens in contact with the eye, the contact lens including a diffractive structure for extending depth of focus of visible light along an optical axis of the contact lens.

As disclosed herein, a contact lens usable during ophthalmic surgery, such as vitreoretinal surgery, includes a diffractive structure that extends depth of focus along an optical axis of the contact lens.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A contact lens for performing ophthalmic surgery, the contact lens comprising:
a diffractive structure for extending depth of focus of visible light along an optical axis of the contact lens.

2. The contact lens of claim 1, wherein the contact lens is selected from a plano-convex lens and a wide angle lens.

3. The contact lens of claim 1, wherein the diffractive structure is formed on an external surface of the contact lens.

4. The contact lens of claim 3, wherein the diffractive structure is formed on a mating surface of the contact lens that mates with an eye during ophthalmic surgery.

5. The contact lens of claim 1, wherein the contact lens comprises a doublet lens, and wherein the diffractive structure is formed on an interior surface of the doublet lens.

6. The contact lens of claim 1, wherein a focal region of the diffractive structure corresponds to the distance between the contact lens and the retina of an eye when the contact lens is in contact with the eye.

7. The contact lens of claim 1, wherein the contact lens includes optical correction for spherical aberration.

8. The contact lens of claim 1, wherein the contact lens includes optical correction for chromatic aberration.

9. A method for performing ophthalmic surgery, comprising:
positioning a first optical axis of a surgical microscope along a second optical axis of an eye of a patient; and
viewing an interior portion of the eye using a contact lens in contact with the eye, wherein the contact lens includes a diffractive structure for extending depth of focus of visible light along an optical axis of the contact lens.

10. The method of claim 9, wherein the contact lens is selected from a plano-convex lens and a wide angle lens.

11. The method of claim 9, wherein the diffractive structure is formed on an external surface of the contact lens.

12. The method of claim 11, wherein the diffractive structure is formed on a mating surface of the contact lens that mates with an eye during ophthalmic surgery.

13. The method of claim 9, wherein the contact lens comprises a doublet lens, and wherein the diffractive structure is formed on an interior surface of the doublet lens.

14. The method of claim 9, wherein a focal region of the diffractive structure corresponds to the distance between the contact lens and the retina of an eye when the contact lens is in contact with the eye.

15. The method of claim 9, wherein the contact lens includes optical correction for spherical aberration.

16. The method of claim 1, wherein the contact lens includes optical correction for chromatic aberration.
